(19) United States
(12) Patent Application Publication
(10) Pub. No.: US 2004/0136955 A1
(43) Pub. Date: Jul. 15, 2004

(54) Modified asialo-interferons and uses thereof

(76) Inventors: Nicholas P. Barker, Southborough, MA (US); Daniel K. Podolsky, Wellesley, MA (US)

Correspondence Address: CLARK & ELBING LLP, 101 FEDERAL STREET, BOSTON, MA, 02110, US

(21) Appl. No.: 10/654,796

(22) Filed: Sep. 3, 2003

Related U.S. Application Data
(60) Provisional application No. 60/408,361, filed on Sep. 5, 2002.
(60) Provisional application No. 60/431,148, filed on Dec. 5, 2002.

(51) Int. Cl. A61K 38/21; C07K 14/56

(52) U.S. Cl. 424/85.4; 530/351

(57) ABSTRACT

The present invention features methods for preparing and using modified asialo-interferons for the treatment of hepatic diseases. Asialo-interferons are modified by the addition of water soluble polymers including, for example, polyethylene glycol (PEG), polyvinylpyrrolidone (PVP), poly(vinyl alcohol) (PVA), poly (alkylene oxides), such as poly (propylene glycol) (PPG), polytrimethylene glycol (PTG), and poly(oxyethylated polyols), such as poly(oxyethylated sorbitol), poly(oxyethylated glycerol, and poly(oxyethylated glucose). The asialo-interferons that may be modified and used for the treatment of hepatic diseases include, for example, asialo-interferon -α, -β, and -γ.

### CROSS-REFERENCE TO RELATED APPLICATIONS

This application claims benefit of the filing date of the co-pending U.S. Provisional Application Nos. 60/408,361 (filed Sep. 5, 2002) and 60/431,148 (filed Dec. 5, 2002), hereby incorporated by reference.

### FIELD OF THE INVENTION

The invention relates to the treatment of hepatic disorders using interferons.

### BACKGROUND OF THE INVENTION

Interferons are a group of naturally-occurring proteins that were first discovered as a result of their ability to prevent viral replication. Additional research has determined that interferons have anti-proliferative effects and are useful in fighting some types of cancer cells. In particular, interferons, including members of the interferon -α, -β, and -γfamily, have been shown to be clinically effective against a number of viral and oncological indications including hepatitis, hairy cell leukemia, chronic myelogenous leukemia, melanoma, follicular lymphoma, and chronic granulomatous disease.

Hepatitis B (HBV) and hepatitis C (HCV) virus infection is a worldwide health problem. More than 350 million people are affected by HBV, making it the most common severe chronic viral infection in the world. Moreover, HBV is the leading cause of liver cancer worldwide. In addition, approximately 170 million people are chronically infected with HCV worldwide, including at least 3.9 million people in the United States. HCV accounts for 30% of end-stage liver disease and liver cancer, and is the leading disease that causes patients to require a liver transplant. However, the treatment options for both HBV and HCV have limited effectiveness, may rapidly lose their effectiveness, and are often poorly tolerated by patients.

In the United States, the incidence of primary liver cancer increased by 71% between 1975 and 1995, and the number of patients diagnosed with liver cancer each year continues to rise. In 2002, the American Cancer Society estimates that 16,600 new cases of primary liver cancer and bile duct cancer will be diagnosed in the United States and that 14,100 Americans will die from the disease.

While interferons are powerful therapeutic compounds, they are rapidly cleared from a patient, necessitating frequent administration to maintain a therapeutically effective level of the compound. Moreover, interferons are not targeted to a particular tissue and, therefore, require relatively high systemic concentrations to achieve a therapeutically effective concentration at the target site. These properties of interferon increase the likelihood of harmful side-effects occurring as a result of the therapy. Accordingly, there is a need to target an interferon to the site of the disease for an extended period of time to maximize the efficacy and minimize the side-effects.

### SUMMARY OF THE INVENTION

In one aspect, the invention features a substantially pure modified mammalian (e.g., human) asialo-interferon which is conjugated to a water soluble polymer having an average molecular weight of approximately 1,000 to 60,000 daltons, 1,000 to 5,000, 5,001 to 10,000, 10,001 to 20,000, 20,001 to 35,000, or 35,001 to 60,000 daltons. The water soluble polymers may be linear or branched and may be internally crosslinked. Preferably, the water soluble polymers are polyethylene glycol (PEG), polyvinylpyrrolidone (PVP), poly(vinyl alcohol) (PVA), poly (alkylene oxides), such as poly (propylene glycol) (PPG), polytrimethylene glycol (PTG), and poly(oxyethylated polyols), such as poly(oxyethylated sorbitol), poly(oxyethylated glycerol, and poly(oxyethylated glucose). The asialo-interferons of this invention may be modified at one, two, three, or more amino acid residues. For asialo-interferons that are modified at more than one amino acid residue, they may be modified using the same or different water soluble polymers. In desirable embodiments, the asialo-interferon is modified at a cysteine, lysine, serine, threonine, tyrosine, aspartic acid, or glutamic acid residue; at a C-terminal carboxyl; or at an N-terminal amine. In a most desirable embodiment, the asialo-interferon is modified at a cysteine or a lysine. Asialo-interferons suitable for modification include, for example, human asialo-interferon-α, asialo-interferon-β, and asialo-interferon-γ. The invention also provides pharmaceutical compositions containing a modified mammalian asialo-interferon and a pharmaceutically acceptable excipient.

In another aspect, the invention features a method of treating a patient with a hepatic disorder by administering an effective amount of a pharmaceutical composition containing a modified mammalian (e.g., human) asialo-interferon. Hepatic disorders amenable to treatment using this method include, for example, viral hepatitis (e.g., infection with the hepatitis B and/or hepatitis C virus), fibrosis of the liver, and hepatic cancers such as diffuse-type hepatocellular carcinoma, febrile-type hepatocellular carcinoma, and cholestatic hepatocellular carcinoma, hepatoblastoma, hepatoid adenocarcinoma, and focal nodular hyperplasia. In desirable embodiments of this aspect of the invention, the modified asialo-interferon is any one of those described in the foregoing aspects. Therapeutically effective amounts of modified asialo-interferons may be, for example, in the range of about 0.025 μg/kg to 10.0 μg/kg body weight (e.g., about 0.025, 0.035, 0.05, 0.075, 0.1, 0.25, 0.5, 1.0, 1.5, 2.0, 2.5, 3.0, or 3.5 μg/kg of body weight). Furthermore, the therapeutically effective amount may be, for example, administered daily, every other day, twice weekly, weekly, every other week, or monthly.

By “interferon” is meant the family of highly homologous species—specific proteins known as interferons, that inhibit viral replication and cellular proliferation and modulate immune response and are substantially identical to interferon-α, -β, or -γ, or biologically active fragments thereof. Methods for evaluating the biological activity of interferon are widely known (e.g., Monkarsh et al., Anal. Biochem. 247:434-440, 1997; Grace et al., J Interferon Cytokine Res. 21: 1103-1115, 2001; Bailon et al., Bioconj. Chem. 12: 195-202, 2001; Pepinsky et al., J Pharmacol. Exp. Therap. 297:1059-66, 2001). Human interferons are grouped into three classes based on their cellular origin and molecular structure: interferon-α (leukocytes), interferon-β (fibroblasts), and interferon-γ (lymphocytes).

By “interferon-α” is meant a protein containing an amino acid sequence that is substantially identical to the interferon-α2 mature polypeptide (amino acids 24-188 of Accession No:P01563; SEQ ID NO:1), or a biologically active fragment thereof. Thus, interferon-α includes the interferon-α2 precursor polypeptide (Accession No:P 01563; SEQ ID NO: 1) and fragments that retain the biological activity of mature interferon-α (e.g., anti-proliferative activity). Also included in this definition are the variant forms of interferon-α2 including, for example, interferon-α2b (R46K mutation of SEQ ID NO: 1) and interferon-α2c (R57H mutation of SEQ ID NO: 1). Interferon-α2b is an O-linked glycoprotein. Interferon-α14c is a N-linked glycoprotein that is glycosylated at Asn-72. Natural interferon is commercially available under the name of Wellferon (Glaxo-SmithKline), Alferon (Interferon), Sumiferon (Sumitomo) and Multiferon (Viragen). Non-glycosylated interferon-α is also commercially available including, for example, recombinant interferon-α2a,under the name Roferon®-A (Roche), recombinant interferon-α2b, under the name Intron®-A (Schering Plough), and recombinant interferon-α2c, under the name of Berofor alpha 2 (Boehringer Ingelheim). Recombinant consensus interferon-con 1 is available under the name of Infergen (Amgen). Of course, prior to use in the composition and methods of this invention, any non-glycosylated interferon must be glycosylated with an oligosaccharide having a terminal galactose residue.

By “interferon-β” is meant a protein containing an amino acid sequence that is substantially identical to the mature interferon-β, polypeptide (amino acids 22-187 of Accession No:P01574; SEQ ID NO:2), or a biologically active fragment thereof. Thus, interferon-β includes, in addition to the mature interferon-β protein that does not contain the signal peptide, the interferon-β precursor polypeptide (Accession No:P01574; SEQ ID NO:2) that does contain the signal peptide, and fragments thereof having the biological activity of interferon-β (e.g., anti-proliferative activity). Interferon-β is a glycoprotein that is glycosylated at Asn80 of the mature interferon-β protein. Recombinant forms of interferon-β have been developed and are commercially available. Interferon-β1a is available under the name Avonex® (Biogen) and Rebif® (Serono). Interferon-β1b is available under the name of Betaseron (Berlex).

By “interferon-γ” is meant a protein containing an amino acid sequence that is substantially identical to the mature interferon-γ polypeptide (amino acids 21-166 of Accession number P01579; SEQ ID NO:3), or a biologically active fragment thereof. Thus, interferon-γ proteins include, in addition to the mature interferon-γ polypeptide that does not contain the signal peptide, the interferon-γ precursor protein (Accession number P01579; SEQ ID NO:3) that contains the signal peptide, and fragments thereof having the biological activity of interferon-γ (e.g., antiproliferative activity). Interferon-γ is glycosylated at Asn48 and, in the dimer, at Asn120. Interferon-γ is commercially available under the name Actimmune® (InterMune).

For any of the aforementioned interferons, variant forms in which one amino acid of the interferon polypeptide sequence has been replaced by another, without losing biological activity, are also included in their definitions. One example would be an interferon-α, -β, or -γ in which a serine or threonine residue is replaced with a cysteine residue, with the cysteine residue later used for conjugating other moieties (e.g., PEG moieties) to the interferon. In such an example, the cysteine is substituted at a position in the interferon molecule such that it does not interfere with folding and is also at least partly exposed on the surface of the molecule.

By “asialo-interferon” is meant a glycosylated interferon lacking a terminal sialic group that is present in the native glycosylated interferon. Removal of the terminal sialic acid residue exposes the underlying galactose moiety. It is the terminal galactose that is recognized by the asialoglycoprotein receptor. Preferably, asialo-interferon contains at least 50%, 70%, 80%, 90%, or even 95% of the carbohydrate moieties present in the native interferon. Most preferably, asialo-interferon lacks only the terminal sialic acid residue. Asialo-interferons can be produced by removing one or more sialic acid groups from a glycosylated interferon, such as interferon-α, -β, or -γ. This removal may be accomplished, for example, by mild acid hydrolysis, or treatment of native glycosylated interferon, such as interferon-α, -β, or -γ, with purified neuroaminidase. For interferons containing more than one sugar chain, selective desialylation may be accomplished using specific neuroaminidase (sialidase) enzymes. Specifically excluded by this definition are completely deglycosylated interferons, including interferons that are typically produced by prokaryotic cells and interferons produced by eukaryotic cells and enzymatically or chemically deglycosylated. Of course, because the goal of removing the sialic acid residue is to create a glycosylated interferon having at least one terminal galactose residue on an oligosaccharide chain, a terminal galactose residue may be engineered by any other appropriate means including, for example, covalently attaching an oligosaccharide to a deglycosylated interferon.

By a “modified asialo-interferon” is meant an asialo-interferon that is conjugated to at least one water-soluble polymer and that retains at least 5%, 10%, 20%, 30%, 40%, 50%, 60%, 70%, 80%, 90%, or 95% of a biological activity of native interferon (e.g., anti-proliferative or anti-viral activity). Examples of water-soluble polymers that may be conjugated to an asialo-interferon include polyalkyl glycols such as polyethylene glycol (PEG), polyvinylpyrrolidone (PVP), poly(vinyl alcohol) (PVA), poly(alkylene oxides) such as poly(propylene glycol) (PPG), polytrimethylene glycol (PTG), and poly(oxyethylated polyols) such as poly(oxyethylated sorbitol), poly(oxyethylated glycerol), and poly(oxyethylated glucose). Desirably, a water-soluble polymer has an average molecular weight of approximately 100 daltons to 200,000 daltons, for example, 100 to 999, 1,000 to 5,000, 5,001 to 10,000, 10,001 to 20,000, 20,001 to 35,000, 35,001 to 60,000, 60,001 to 100,000, or 100,001 to 200,000 daltons. In more desirable embodiments, a water-soluble polymer has an average molecular weight of approximately 1,000 to 5,000, 5,001 to 10,000, 10,001 to 20,000, 20,001 to 35,000, 35,001 to 60,000, or 60,001 to 100,000 daltons. In addition, also included in this definition are forms of these polymers that have been activated using a method described herein.

The modified asialo-interferon may be modified, for example, by covalently attaching a polymer at a cysteine, lysine, serine, threonine, tyrosine, aspartic acid, or glutamic acid residue; at a C-terminal carboxyl; or at an N-terminal amine of the interferon. In other desirable embodiments, a modified asialo-interferon has been modified by the conjugation of a water-soluble polymer to more than one amino acid residue. One skilled in the art readily would be able to determine the most desirable residues to conjugate a water-soluble polymer to an asialo-interferon and with which average molecular weight of the polymer, for example, by measuring and comparing the relative anti-viral activity, anti-proliferative activity, or biodistribution/pharmacokinetics of each positional isomer of modified asialo-interferon. In addition, a combination of several isomers may be used to give a composite pharmacokinetic profile. For instance, these activities may be determined by using an anti-viral or anti-proliferative assay described herein.

By “pegylated asialo-interferon” is meant an asialo-interferon that is conjugated to at least one polyethylene glycol (PEG) polymer and that retains at least 5%, 10%, 20%, 30%, 40%, 50%, 60%, 70%, 80%, 90%, or 95% of a biological activity of native interferon (e.g., anti-proliferative or anti-viral activity). For example, the PEG may be a monomethoxy PEG (mPEG) and it may be covalently attached to an asialo-interferon. Desirably, the PEG is an mPEG polymer having an average molecular weight of, for example, about 1,000 to 5,000, 5,001 to 10,000, 10,001 to 20,000, 20,001 to 35,000, or 35,001 to 60,000 daltons (e.g., 1,000, 1,450, 3,350, 5,000, 6,000, 8,000, 10,000, 12,000, 20,000, 30,000, 35,000, 40,000, or 60,000 daltons). In more desirable embodiments, the mPEG polymer has an average molecular weight of, for example, about 5,000, 12,000, 20,000, or 40,000 daltons. The pegylated asialo-interferon may be pegylated, for example, at a cysteine, lysine, serine, threonine, tyrosine, aspartic acid, or glutamic acid residue; at a C-terminal carboxyl; or at an N-terminal amine of the interferon. In other desirable embodiments, a pegylated asialo-interferon is pegylated at more than one amino acid residue. One skilled in the art readily would be able to determine the most desirable residues to pegylate in an asialo-interferon and with which average molecular weight of PEG, for example, by measuring and comparing the relative anti-viral activity, anti-proliferative activity, or biodistribution/pharmacokinetics of each positional isomer of pegylated asialo-interferon. In addition, a combination of several isomers may be used to give a composite pharmacokinetic profile. For instance, these activities may be determined by using an anti-viral or anti-proliferative assay described herein.

By “pvpylated asialo-interferon” is meant an asialo-interferon that is conjugated to at least one polyvinylpyrrolidone (PVP) molecule and that retains at least 5%, 10%, 20%, 30%, 40%, 50%, 60%, 70%, 80%, 90%, or 95% of a biological activity of native interferon (e.g., anti-proliferative or anti-viral activity). The pvpylated asialo-interferon may be pvpylated, for example, at a cysteine, lysine, serine, threonine, tyrosine, aspartic acid, or glutamic acid residue; at a C-terminal carboxyl; or at an N-terminal amine of the interferon. In other desirable embodiments, a pvpylated asialo-interferon is pvpylated at more than one amino acid residue. In a particularly useful embodiment, the PVP polymer has an average molecular weight of about 17,000 daltons.

One skilled in the art readily would be able to determine the most desirable residues to pvpylate in an asialo-interferon and with how many PVP molecules, for example, by measuring and comparing the relative anti-viral activity, anti-proliferative activity, or biodistribution/pharmacokinetics of each positional isomer of pvpylated asialo-interferon. In addition, a combination of several isomers may be used to give a composite pharmacokinetic profile. For instance, these activities may be determined by using an anti-viral or anti-proliferative assay described herein.

By a “hepatic disorder” is meant any disease affecting a tissue or cell of the liver. Examples of a “hepatic disorder” include viral hepatitis, hepatic cancer, and fibrosis of the liver. Hepatitis may be caused by, for example, an infection of the liver by a hepatitis B or a hepatitis C virus. An infection by a hepatitis B or a hepatitis C virus may be diagnosed by one skilled in the art using standard methods, e.g., by determining if the patient has antibodies against a hepatitis virus, or by the presence of viral RNA.

By a “hepatic cancer” is meant any disorder in which a tissue or cell of the liver undergoes abnormal proliferation. Liver cells that may give rise to hepatic cancer include cells of the bile ducts, blood vessels, such as the portal vein, dendritic cells, or hepatocytes. Hepatic cancers include, but are not limited to, hepatocellular carcinoma, such as diffuse-type hepatocellular carcinoma, febrile-type hepatocellular carcinoma, and cholestatic hepatocellular carcinoma, hepatoblastoma, hepatoid adenocarcinoma, and focal nodular hyperplasia. In addition, hepatic cancers may be the result of a chronic infection by a hepatitis virus.

Patients whose hepatic cancer expresses an asialoglycoprotein receptor are amenable to treatment with a modified asialo-interferon; these patients may be identified using diagnostic methods that are standard in the art (e.g., Burgess et al., Hepatology 15:702-706, 1992; Hirose et al., Biochem. and Biophys. Research Comm. 287:675-681, 2001; Hyodo et al., Liver 13:80-5, 1993; Trere et al., Br. J Cancer 81:404-8, 1999).

By “antineoplastic therapy” is meant any medical procedure or treatment used to inhibit, partially or completely, the proliferation of a neoplasm. Typically, antineoplastic therapies include surgical procedures that remove some or all of the neoplastic cells from the patient (e.g., hepatectomy), radiation therapy, and chemotherapy. Particularly useful classes of antineoplastic chemotherapeutics that can be administered in combination with the asialo-interferons according to the present invention include, for example, alkylating agents, antimetabolites, nitrosoureas, and plant alkaloids. Desirably, “antineoplastic therapy” results in, for example, a 25%, 50%, or 75% reduction in the proliferation of a neoplasm. In more desirable embodiments, “antineoplastic therapy” results in, for example, an 80%, 90%, 95%, or even 99% reduction in proliferation of the neoplasm. Examples of antineoplastic agents that may be used in combination with a modified asialo-interferon are described, for instance, in Wadler and Schwartz (Cancer Res. 50:3473-3486, 1990).

By an “anti-viral agent” is meant any compound that destroys a virus or that reduces a virus's ability to replicate or disseminate in vivo. Examples of anti-viral agents include interferon-α, -β, -γ, ribavirin (1β-D ribofuranosyl-1H-1, 2,4 triazole 3-carboxamide) and its derivatives, and the synthetic nucleotide analog lamivudine ((cis-1-[2′-Hydroxymethyl-5′-(1,3-oxathiolanyl)] cytosine) and its analogs. In addition, one skilled in the art would know how to assay the anti-viral activity of an agent using standard methods (e.g., the methods disclosed in Monkarsh et al., Analytical Biochemistry 247:434-440, 1997; Bailon et al., Bioconjugate Chem. 12:195-202, 2001; and Grace et al., J. of Interferon and Cytokine Research 21:1103-1115, 2001) and those described herein. Desirably, an “anti-viral agent” results in a reduction in viral replication or dissemination of, for example, at least 10%, 20%, 30%, or 50%. In more desirable embodiments, an anti-viral agent reduces viral replication or dissemination, for example, by 70%, 80%, 90%, 95%, or even 99%.

By “asialoglycoprotein receptor-expressing hepatic disorder” is meant any hepatic disorder that contains cells expressing detectable levels of the asialoglycoprotein receptor protein (Accession No.: NP_{—}001662 or P07307) or proteins substantially identical to the asialoglycoprotein receptor, or nucleic acids. The cells may be assessed for asialoglycoprotein receptor expression using any appropriate in vivo, ex vivo, or in vitro technique. For example, cells extracted from a patient during a biopsy or surgical resection can be characterized for asialoglycoprotein receptor expression using standard immunohistochemistry, Northern, or Western blotting techniques, or an ELISA. In addition, asialoglycoprotein receptors are known to the skilled artisan and are described, for example, in Spiess et al. (Proc. Natl. Acad. Sci. USA 82:6465-6469, 1985) and Spiess et al. (J. Biol. Chem. 260:1979-1982, 1985).

By “substantially identical” is meant a polypeptide or nucleic acid exhibiting at least 75%, but preferably 85%, more preferably 90%, most preferably 95%, or even 99% identity to a reference amino acid or nucleic acid sequence . For polypeptides, the length of comparison sequences will generally be at least 20 amino acids, preferably at least 30 amino acids, more preferably at least 40 amino acids, and most preferably 50 amino acids. For nucleic acids, the length of comparison sequences will generally be at least 60 nucleotides, preferably at least 90 nucleotides, and more preferably at least 120 nucleotides.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By “an effective amount” is meant an amount of a compound, alone or in a combination according to the invention, required to inhibit the growth of a neoplasm or to prevent viral replication or dissemination in vivo. The therapeutically effective amount of active compound(s) used to practice the present invention for therapeutic treatment of neoplasms (i.e., cancer) and viral infection varies depending upon the manner of administration, the age, body weight, and general health of the subject. Ultimately, the attending physician will decide the appropriate amount and dosage regimen. Such an amount is referred to as a “therapeutically effective” amount.

“An effective amount” of a modified asialo-interferon may be, for example, in the range of about 0.0035 μg to 20 μg/kg body weight/day or 0.010 μg to 140 μg/kg body weight/week. Desirably, “a therapeutically effective amount” is in the range of about 0.025 μg to 10.0 μg/kg, for example, about 0.025, 0.035, 0.05, 0.075, 0.1, 0.25, 0.5, 1.0, 1.5, 2.0, 2.5, 3.0, 3.5, 4.0, 5.0, 6.0, 7.0, 8.0, or 9.0 μg/kg body weight administered daily, every other day, twice weekly, or weekly. Furthermore, “a therapeutically effective amount” of a modified asialo-interferon may be, for example in the range of about 100 μg/m² to 100,000 μg/m² administered daily, every other day, twice weekly, weekly, every other week, or once a month. In a desirable embodiment, the therapeutically effective amount is in the range of about 1,000 μg/m² to 20,000 μg/m², for example, about 1,000, 1,500, 4,000, or 14,000 μg/m² of a modified asialo-interferon administered daily, every other day, twice weekly, weekly, every other week, or once a month.

By “fragment” is meant a portion of a protein or nucleic acid that is substantially identical to a reference protein or nucleic acid, and retains at least 50% or 75%, more preferably 80%, 90%, or 95%, or even 99% of the biological activity (e.g., the anti-neoplastic or anti-viral activity) of the reference protein or nucleic acid, as may be determined by using an anti-viral or anti-neoplastic assay described herein.

The modified asialo-interferons of the present invention provide numerous advantages over naturally-occurring forms of interferon for treating disease. The advantages of modification (e.g., pegylation and pvpylation) include: increased solubility, reduced renal and immunoclearance, reduced proteolytic susceptibility, and reduced immunogenicity. As described herein, modification of an asialo-interferon, aids in reducing the rate at which the compound is eliminated from the body and thereby increases the therapeutic effectiveness of the compound. As a modified compound is present in the body for a longer time period than its non-modified counterpart, less of a modified compound may be administered to a patient while achieving the same therapeutic result. Moreover, the modified asialo-interferons target the liver which may result in a reduced occurrence of secondary effects that may be associated with administration of unmodified interferons and that are not beneficial in the treatment is also reduced.

In addition, removing the sialic acid group from an interferon exposes its terminal galactose residues and the asialo-interferon is thereby targeted to any cell expressing an asialoglycoprotein receptor. It has been demonstrated that the total number of receptor sites in a liver is increased from 140,000 (+/−65,000) sites per cell in a normal liver to 300,000 (+/−125,000) sites per cell in a liver affected by fibrosis, chirrhosis, or hepatocarcinoma (Eisenberg et al., J Hepatol. 13:305-309, 1991). In view of these findings, a modified asialo-interferon would be preferentially targeted to the liver. Such targeting increases the local concentration of the therapeutic compound at the treatment site, further enabling a reduction in the dosage needed to effectively treat a disorder. Accordingly, the compounds of the present invention have an increased therapeutic effectiveness due to increased retention of the therapeutic compound and targeting of particular tissues.

### BRIEF DESCRIPTION OF THE DRAWINGS

FIG. 1 is a schematic illustration of the structure of natural human interferon-β. Also illustrated are the cleavage sites of typical biantennary complex-type sugar chains of natural human interferon-β by neuraminidase. Abbreviations: Fuc, fucose; GlcNAc, N-acetylglucosamine; Man, mannose; Gal, galactose; NeuAc, N-acetylneuraminic acid (sialic acid).

FIG. 2A is the amino acid sequence of a human interferon-α-2 precursor polypeptide (Accession No.:P01563) (SEQ ID NO:1), including the signal peptide (amino acids 1-23; bold text). The mature interferon-α-2 polypeptide (plain text) extends from amino acid 24-188. The underlined threonine at amino acid 129 is the site of O-linked glycosylation.

FIG. 2B is the nucleic acid sequence (Accession No.: NM_{—}000605) (SEQ ID NO:4) of an mRNA that encodes human interferon-α-2 precursor polypeptide. The coding sequence extends from nucleic acid 69 to nucleic acid 635. The start and stop codons are underlined. Several variant forms of this nucleic acid sequence exist, which include the following nucleic acid changes: A to G at nucleic acid position 205; A to G at nucleic acid position 667; C to T at nucleic acid position 909; and/or A to G at nucleic acid position 949.

FIG. 3A is the amino acid sequence of a human interferon-β precursor polypeptide (Accession No.:P01574) (SEQ ID NO:2), including the signal peptide (amino acids 1-21; bold text). The mature human interferon-β polypeptide (plain text) extends from amino acid 22-187. The underlined asparagine at amino acid position 101 is the site of N-linked glycosylation. A human interferon-β variant polypeptide contains a tyrosine at amino acid position 162 (C to Y).

FIG. 3B is the nucleic acid sequence (Accession No:NM₁₃002176) (SEQ ID NO:5) of an mRNA that encodes human interferon-β precursor polypeptide. The coding sequence extends from nucleic acid 1-564. The start and stop codons are underlined. Several variant forms of this nucleic acid sequence exist, which include the following nucleic acid changes: C to T at nucleic acid position 153 and C to T at nucleic acid position 228.

FIG. 4A is the amino acid sequence of a human interferon-γprecursor protein (Accession No.:P01579) (SEQ ID NO:3) including the signal peptide (amino acids 1-20; bold text). The mature human interferon-γ polypeptide (plain text) extends from amino acid 21-166. The underlined asparagines at amino acid positions 48 and 120 of the interferon-γ precursor protein are the site of N-linked glycosylation (although Asn120 is only glycosylated in the dimer).

FIG. 4B is the nucleic acid sequence of an MRNA that encodes human interferon-γ precursor protein (NM₁₃000619) (SEQ ID NO:6). The coding sequence extends from nucleic acid 109-609. The start and stop codons are underlined. Several variant forms of this nucleic acid sequence exist, which include the following nucleic acid changes: A to G at nucleic acid 624; A to G at nucleic acid 705; A to T at nucleic acid 732; C to T at nucleic acid 789; C to T at nucleic acid 986; and A to G at nucleic acid 1148.

### DETAILED DESCRIPTION

The present invention features modified asialo-interferons, e.g., pegylated asialo-interferons and pvpylated asialo-interferons, as well as methods of using such compounds for treating neoplastic disorders and viral infections. Modified asialo-interferons are targeted to cells expressing the asialoglycoprotein receptor and an interferon receptor. Accordingly, such compounds may be used to treat neoplasms or viral infections of cells expressing either of these receptors; nevertheless, the optimal activity will be exerted to cells expressing both receptors.

Removing the sialic acid group from an interferon exposes its terminal galactose residues and the asialo-interferon is thereby targeted to any cell expressing an asialoglycoprotein receptor. It has been demonstrated that the total number of receptor sites in a liver is increased from 140,000 (+/−65,000) sites per cell in a normal liver to 300,000 (+/−125,000) sites per cell in a liver affected by fibrosis, chirrhosis, or hepatocarcinoma (Eisenberg et al., J Hepatol. 13:305-309, 1991). In view of these findings, a modified asialo-interferon would be preferentially targeted to the liver. Such targeting increases the local concentration of the therapeutic compound at the treatment site, further enabling a reduction in the dosage needed to effectively treat a disorder. Accordingly, the compounds of the present invention have an increased therapeutic effectiveness due to increased retention of the therapeutic compound and targeting of particular tissues.

The modified asialo-interferons of the present invention provide numerous advantages over naturally-occurring forms of interferon for treating disease. The advantages of modification (e.g., pegylation and pvpylation) include: increased solubility, reduced renal and immunoclearance, reduced proteolytic susceptibility, and reduced immunogenicity. As described herein, modification of an asialo-interferon, aids in reducing the rate at which the compound is eliminated from the body and thereby increases the therapeutic effectiveness of the compound. As a modified compound is present in the body for a longer time period than its non-modified counterpart, less of a modified compound may be administered to a patient. By reducing the dosage, the potential occurrence of secondary effects that may be associated with administration of the compound and that are not beneficial in the treatment is also reduced.

Modified Asialo-Interferon Therapy

Like native interferon, modified asialo-interferon may be used to treat hepatic diseases including hepatitis and some cancers. For example, hepatitis B and C, and asialoglycoprotein-expressing hepatic cancers may be treated in a mammal (e.g., a human) by administering to the mammal a pharmaceutical composition that includes a therapeutically effective amount of a modified asialo-interferon (e.g., modified asialo-interferon-α, modified asialo-interferon-β, or modified asialo-interferon-γ) using the methods described herein.

In addition, modified asialo-interferons may be used in combination with other therapeutic approaches such as chemotherapy, radiation therapy, surgical intervention, and the administration of additional anti-viral compounds. (Such combinations are standard in the art and are described, for example in Wadler et al. (Cancer Res. 50:3473-86, 1990).) For instance, modified asialo-interferon may be administered with a therapeutically effective amount of ribavirin (1 β-D ribofuranosyl-1 H-1, 2,4 triazole 3-carboxamide), or a derivative thereof, to treat viral infections. Alternatively, modified asialo-interferon may be administered with a therapeutically effective amount of lamivudine ((cis-1-[2′-Hydroxymethyl-5′-(1,3-oxathiolanyl)] cytosine), or a lamivudine analog, to treat viral infections.

Asialoglycoprotein Receptor

The asialoglycoprotein receptor is a transmembrane protein that is present at high density (50,000 to 500,000 sites/cell) on hepatocytes and mediates the binding and internalization of extracellular glycoproteins having exposed terminal galactose residues. The asialoglycoprotein receptor is a low affinity receptor, and it's affinity for ligand varies with the number of galactose clusters present on the ligand (Lee et al., J Biol. Chem. 258:199-202, 1983). The receptor has a lower affinity for ligand having clusters of two galactose residues, biantennary (K_{D}˜10⁻6), than for ligand having clusters of three galactose residues, triantennary (K_{D}˜10⁻⁸−10⁻9).

Delivery of Interferons

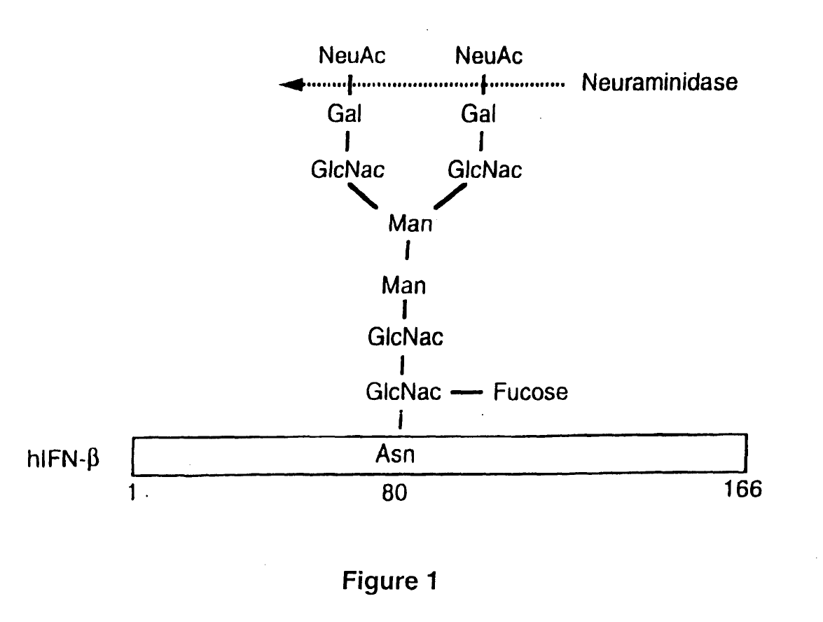

Removing a sialic acid group from any native interferon exposes the terminal galactose residues (FIG. 1), creating a recognition site for the asialoglycoprotein receptor. This modification imparts the benefit that asialo-interferon is selectively targeted to a tissue expressing an asialoglycoprotein receptor such as the liver. In addition, binding to the asialoglycoprotein receptor and receptor complex internalization likely increases asialo-interferon's ability to activate intracellular interferon-α/β receptor pools. Moreover, targeting asialo-interferon to the asialoglycoprotein receptor likely increases the local concentration of asialo-interferon at the cell surface thus increasing the probability that asialo-interferon will bind to the high affinity interferon-α/β receptors, which are present at low density (100-5,000 sites/cell) on hepatocytes.

Cell Surface Interferon Receptor Binding

Additionally, increasing the local concentration of asialo-interferon on the hepatocyte surface, through binding to the asialoglycoprotein receptor, makes it more likely that an asialo-interferon-α, -β, or -γ will interact with the interferon-α/β receptor or interferon-γ receptor. The transfer of the asialo-interferon from the asialoglycoprotein receptor to the interferon-α/β receptor or interferon-γ receptor is more likely to occur in asialo-interferon compositions having a reduced affinity for the asialoglycoprotein receptor. The affinity of the asialoglycoprotein receptor for ligand varies with the number of galactose clusters present on its ligand (Lee et al., J. Biol. Chem. 258:199-202, 1983). The asialoglycoprotein receptor has a lower affinity for biantennary ligand (K_{D}˜10⁻⁶), than for triantennary ligand (K_{D}˜10⁻⁸ −10⁻⁹).

Various methods are known in the art for creating interferons having different proportions of biantennary complexes. For example, interferons produced by fibroblast cells have a higher proportion of biantennary complexes than interferons produced by CHO cells. In particular, human asialo-interferon-β produced in human fibroblasts contains about 82% biantennary galactose-terminal oligosaccharides and about 18% triantennary galactose-terminal oligosaccharides.

Given the extended conformation of interferon-β's carbohydrate chain (Karpusas et al., Proc. Natl. Acad. Sci USA 94:11813-11818, 1997), interferon-β likely interacts with both the asialoglycoprotein receptor and the interferon-α/β receptor simultaneously. Thus, the abundant asialoglycoprotein receptor may concentrate asialo-interferon-β at the cell surface where it likely interacts simultaneously with the less abundant interferon-α/β receptor.

Intracellular Interferon Receptor Binding

Binding of interferon-α, -β, or -γ to intracellular interferon receptors likely triggers interferon signaling. Interferon-α incorporated into liposomes can produce significantly greater activity than free interferon-α, supporting the hypothesis that interferons do not need to reach the cell surface to exert activity. Furthermore, ligand binding to the asialoglycoprotein receptor triggers internalization of the receptor-ligand complex, providing asialo-interferons with access to intracellular interferon receptors.

Interferon Production

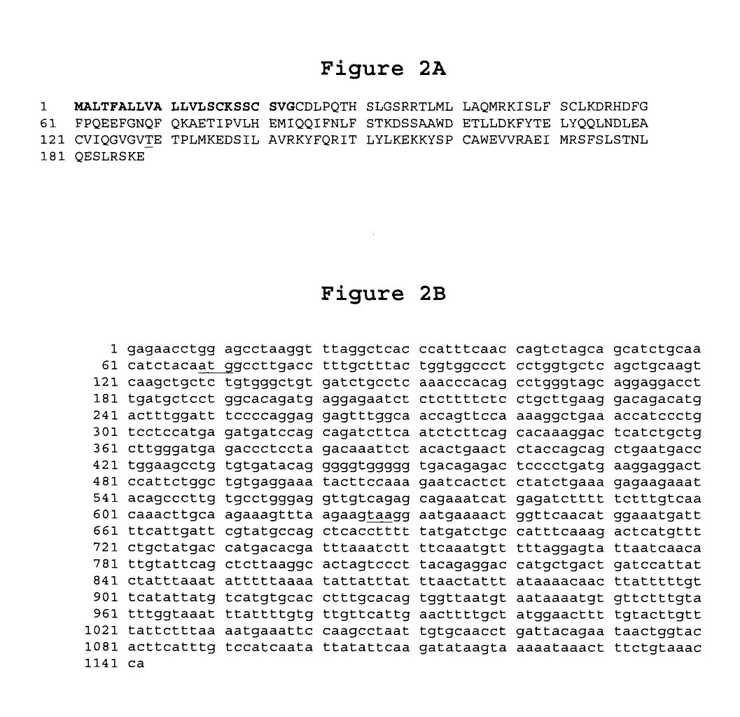

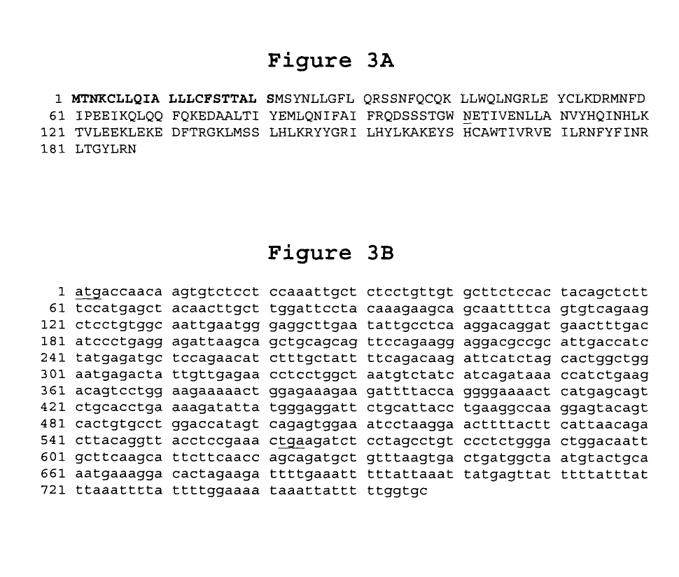

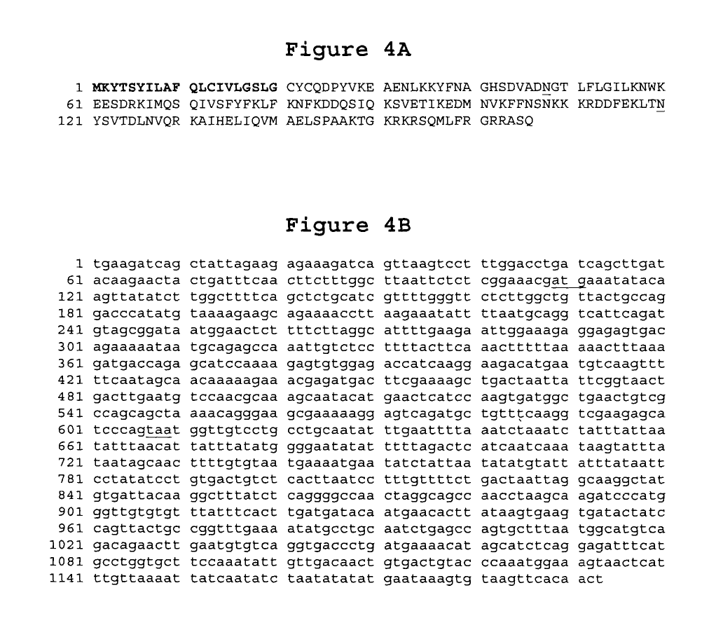

In general, polypeptides of the invention, such as interferon-α (FIG. 2A), -β (FIG. 3A), or -γ (FIG. 4A) may be produced by transformation of a suitable host cell, for example, a eukaryotic cell, with all or part of a polypeptide-encoding nucleic acid molecule, such as the interferon-a encoding nucleic acid shown in FIG. 2B, the interferon-β encoding nucleic acid shown in FIG. 3B, the interferon-γ encoding nucleic acid shown in FIG. 4B or a fragment thereof in a suitable expression vehicle.

Those skilled in the field of molecular biology will understand that any of a wide variety of expression systems may be used to provide the recombinant protein. Eukaryotic interferon peptide expression systems may be generated in which an interferon peptide gene sequence is introduced into a plasmid or other vector, which is then used to transform living cells. Constructs in which the interferon peptide cDNA contains the entire open reading frame inserted in the correct orientation into an expression plasmid may be used for protein expression. Eukaryotic expression systems allow for the expression and recovery of interferon peptide fusion proteins in which the interferon peptide is covalently linked to a tag molecule which facilitates identification and/or purification. An enzymatic or chemical cleavage site can be engineered between the interferon peptide and the tag molecule so that the tag can be removed following purification.

Typical expression vectors contain promoters that direct the synthesis of large amounts of mRNA corresponding to the inserted interferon peptide nucleic acid in the plasmid-bearing cells. They may also include a eukaryotic or prokaryotic origin of replication sequence allowing for their autonomous replication within the host organism, sequences that encode genetic traits that allow vector-containing cells to be selected for in the presence of otherwise toxic interferons, and sequences that increase the efficiency with which the synthesized mRNA is translated. Stable long-term vectors may be maintained as freely replicating entities by using regulatory elements of, for example, viruses (e.g., the OriP sequences from the Epstein Barr Virus genome). Cell lines may also be produced that have integrated the vector into the genomic DNA, and in this manner the gene product is produced on a continuous basis.

The precise host cell used is not critical to the invention. A polypeptide of the invention may be produced in a eukaryotic host (e.g., Saccharomyces cerevisiae, insect cells, e.g., Sf21 cells, or mammalian cells, e.g., NIH 3T3, HeLa, CHO, COS cells, or desirably in fibroblasts). Such cells are available from a wide range of sources (e.g., the American Type Culture Collection, Manassas, Va.; also, see, e.g., Ausubel et al., Current Protocols in Molecular Biology, Wiley Interscience, New York, 2001). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the invention. Mammalian cells, for example, can be used to express an interferon polypeptide. Stable or transient cell line clones can be made using interferon peptide expression vectors to produce the interferon polypeptides in a soluble (truncated and tagged) form. Appropriate cell lines include, for example, COS, HEK293T, CHO, or NIH 3T3 cell lines. Appropriate vectors include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses, and retroviruses, and vectors derived from combinations thereof

Once the appropriate expression vectors are constructed, they are introduced into an appropriate host cell by transformation techniques, such as, but not limited to, calcium phosphate transfection, DEAE-dextran transfection, electroporation, microinjection, protoplast fusion, or liposome-mediated transfection. The host cells that are transfected with the vectors of this invention may include (but are not limited to) yeast, fungi, insect cells (using, for example, baculoviral vectors for expression in SF9 insect cells), or cells derived from mice, humans, or other animals. In vitro expression of interferon polypeptides, fusions, or polypeptide fragments encoded by cloned DNA may also be used. Those skilled in the art of molecular biology will understand that a wide variety of expression systems and purification systems may be used to produce recombinant interferon polypeptides and fragments thereof Some of these systems are described, for example, in Ausubel et al. (Current Protocols in Molecular Biology, John Wiley & Sons, New York, N.Y. (2000), hereby incorporated by reference).

Native, glycosylated interferon can be isolated from human cells, which produce it naturally, or from transgenic eukaryotic cells that have been engineered to express a recombinant interferon gene. Methods for natural or recombinant production of interferon are generally described in U.S. Pat. Nos.: 4,124,702, 4,130,641, 4,680,261, 4,758,510, 5,376,567, 5,795,779, and 5,827,694. Alternatively, isolated and purified human interferon is available commercially (e.g., Sigma Chemical Co. Catalog Nos. I 2396, I 2271,I 1640, and I 6507).

Once the recombinant polypeptide of the invention is expressed, it is isolated, e.g., using affinity chromatography. In one example, an antibody (e.g., produced by standard techniques known to one skilled in the art) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra).

Once isolated, the recombinant protein can, if desired, be further purified, e.g., by high performance liquid chromatography or other chromatographies (see, e.g., Fisher, Laboratory Techniques In Biochemistry And Molecular Biology, eds., Work and Burdon, Elsevier, 1980).

Polypeptides of the invention, particularly short peptide fragments, can also be produced by chemical synthesis (e.g., by the methods described in Solid Phase Peptide Synthesis, 2nd ed., The Pierce Chemical Co., Rockford, Ill., 1984).

These general techniques of polypeptide expression and purification can also be used to produce and isolate useful peptide fragments or analogs that have a biological activity of an interferon described herein.

Asialo-Interferon Production

Various methods are known for creating interferons having differing proportions of biantennary complexes. Interferons produced by fibroblast cells, for example, have a higher proportion of biantennary complexes than interferons produced by Chinese hamster ovary (CHO) cells. Specifically, human asialo-interferon-β produced in human fibroblasts contains about 82% biantennary galactose-terminal oligosaccharides and about 18% triantennary galactose-terminal oligosaccharides.

Asialo-interferon can be produced by removing a terminal sialic residue from interferon which is glycosylated and normally has such a residue by virtue of its having been produced in a eukaryotic cell (see, e.g., U.S. Pat. No. 4,184,917 and references cited therein, and Kasama et al., J Interfer. Cyto. Res. 15:407-415, 1995). The terminal sialic residue can be removed, for example, by mild acid hydrolysis or treatment of native glycosylated interferon with isolated and purified bacterial or viral neuraminidase as described in Drzenieck et al. (Microbiol. Immunol. 59:35, 1972). Purified neuraminidases, including neuraminidases from Clostridium perfringens, Salmonella typhimurium, Arthrobacter ureafaciens, and Vibrio cholerae are readily available from Sigma Chemical Co. (St. Louis, Mo.) (Catalog Nos. N 3642, N 5146, N 7771, N 5271, N 6514, N 7885, N 2876, N 2904, N 3001, N 5631, N 2133, N 6021, N 5254, and N 4883).

For instance, to produce human asialo-interferon-β, 20 mg of insoluble neuraminidase attached to beaded agarose (about 0.22 units) may be suspended in 1 ml distilled water in a microcentrifuge tube and allowed to hydrate briefly. The agarose may be pelleted by centrifugation and washed three times with 1 ml of sodium acetate buffer (pH 5.5) containing 154 mM NaCl and 9 mM calcium chloride and the gel (about 72 μl) may be re-suspended in 150 μl of the sodium acetate buffer. For example, glycosylated human interferon-β (3×10⁶ IU/vial, about 0.15 mg) may be suspended in 150 μl of sodium acetate buffer. The gel and interferon-β can then mixed and incubated on a rotating platform at 37° C. for three hours and the mixture can be separated from the neuraminidase by centrifugal filtration through a 0.2 μm filter. The asialo-interferon may be stored at −80° C. for extended periods of time.

A further exemplary method of preparing asialo-interferon involves digesting natural human interferon-β with one unit of Arthrobacter ureafaciens-derived neuraminidase in 1 ml of 5 mM formic acid (pH 3.5) at 37° C. for three hours. Following hydrolysis, the desialylated interferon-β may be isolated on a C 18 reversed-phase column (e.g., Zorbax®PR-10) with a linear gradient of acetonitrile in 0.1% trifluoroacetic acid. Other methods of producing asialo-interferons are generally described in U.S. Pat. No. 6,296,844 (hereby incorporated by reference).

Preparation of Pegylated Asialo-Interferon

Polyethylene glycol (PEG) is a neutral, water-soluble, non-toxic polymer. (PEG of various average molecular weights is commercially available from Sigma-Aldrich (St. Louis, Mo.), and PEG that has been modified to be amenable to protein conjugation is commercially available from Shearwater Corporation (Huntsville, Ala.) or Valentis, Inc. (Burlingame, Calif.)) The lack of toxicity is reflected in the fact that PEG is one of the few synthetic polymers approved for internal use by the FDA, appearing in food, cosmetics, personal care products and pharmaceuticals. In an aqueous medium, the long chain-like PEG molecule is heavily hydrated and it is in rapid motion. This rapid motion leads to the PEG sweeping out a large volume (its “exclusion volume”) and prevents the approach of other molecules. In a very real sense, PEG is largely invisible to biological systems and is revealed only as moving bound water molecules. One result of this property is that PEG is non-immunogenic.

To affect covalent attachment of the polymer molecule(s) to the polypeptide, the hydroxyl end groups of the polymer molecule must be provided in activated form, i.e. with reactive functional groups (examples of which include primary amino groups, hydrazide (HZ), thiol, succinate (SUC), succinimidyl succinate (SS), succinimidyl succinamide (SSA), succinimidyl proprionate (SPA), succinimidy carboxymethylate (SCM), benzotriazole carbonate (BTC), N-hydroxysuccinimide (NHS), aldehyde, nitrophenylcarbonate (NPC), and tresylate (TRES)). Suitably activated polymer molecules are commercially available, e.g. from Shearwater Polymers, Inc., Huntsville, Ala. U.S.A. Alternatively, the polymer molecules can be activated by conventional methods known in the art, e.g. as disclosed in WO 90/13540. Specific examples of activated linear or branched polymer molecules for use in the present invention are described in the Shearwater Polymers, Inc. 1997 and 2000 Catalogs (Functionalized Biocompatible Polymers for Research and pharmaceuticals, Polyethylene Glycol and Derivatives, incorporated herein by reference). Specific examples of activated PEG polymers include the following linear PEGs: NHS-PEG (e.g. SPA-PEG, SSPA-PEG, SBA-PEG, SS-PEG, SSA-PEG, SC-PEG, SG-PEG, and SCM-PEG), and NOR-PEG), BTC-PEG, EPOX-PEG, NCO-PEG, NPC-PEG, CDI-PEG, ALD-PEG, TRES-PEG, VS-PEG, IODO-PEG, and MAL-PEG, and branched PEGs such as PEG₂-NHS and those disclosed in U.S. Pat. Nos. 5,932,462 and 5,643,575, both of which references are incorporated herein by reference.

Examples of PEG derivatives that may be conjugated to an asialo-interferon include those provided below.

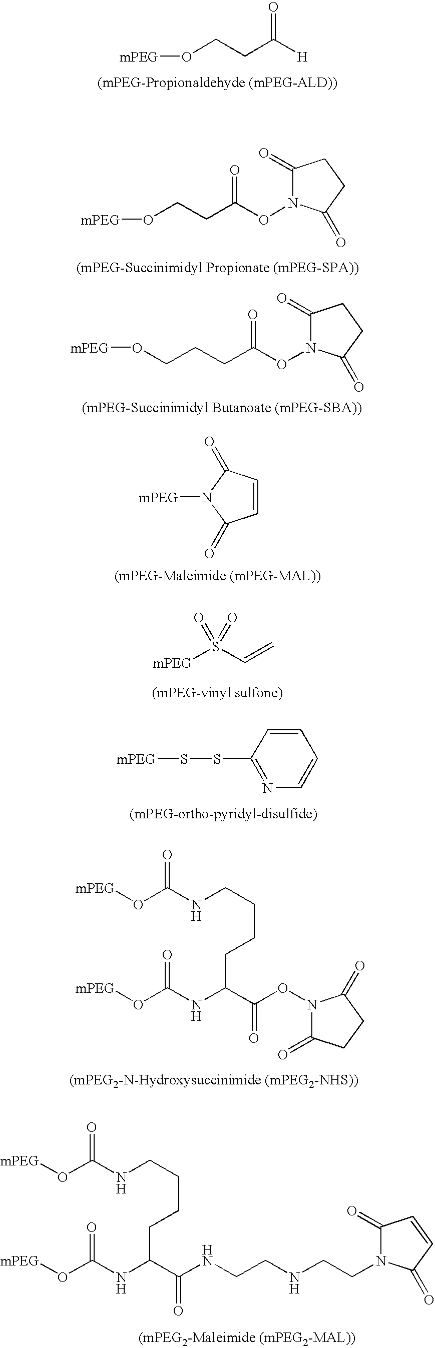

Furthermore, the following publications, incorporated herein by reference, disclose useful polymer molecules and/or PEGylation chemistries: U.S. Pat. Nos. 5,824,778, 5,476,653, WO 97/32607, EP 229,108, EP 402,378, U.S. Pat. Nos. 4,902,502, 5,281,698, 5,122,614, 5,219,564, WO 92/16555, WO 94/04193, WO 94/14758, WO 94/17039, WO 94/18247, WO 94/28024, WO 95/00162, WO 95/11924, WO 095/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. Nos. 5,473,034, 5,516,673, EP 605 963, U.S. Pat. No. 5,382,657, EP 510 356, EP 400 472, EP 183 503 and EP 154 316.

The conjugation of the polypeptide and the activated polymer molecules is conducted by use of any conventional method, e.g. as described in the following references (which also describe suitable methods for activation of polymer molecules):Harris and Zalipsky, eds., Poly(ethylene glycol) Chemistry and Biological Applications, AZC, Washington; R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.). The skilled person will be aware that the activation method and/or conjugation chemistry to be used depends on the attachment group(s) of the interferon polypeptide as well as the functional groups of the polymer (e.g., being amino, hydroxyl, carboxyl, aldehyde for sulfydryl). The PEGylation may be directed towards conjugation to all available attachment groups on the polypeptide (i.e., such attachment groups that are exposed at the surface of the polypeptide) or may be directed towards specific attachment groups, e.g., the N-terminal amino group (U.S. Pat. No. 5,985,265). Furthermore, the conjugation may be achieved in one step or in a stepwise manner (e.g., as described in WO 99/55377).

PEGs that may be conjugated with an asialo-interferon include ones having an average molecular weight of 1,000 to 5,000, 5,001 to 10,000, 10,001 to 20,000, 20,001 to 35,000, or 35,001 to 60,000 daltons (e.g., 3,350, 5,000, 8,000, 10,000, 12,000, 20,000, 30,000, 35,000, 40,000, or 60,000 daltons). Low molecular weight PEGs (e.g., ones having an average molecular weight of 5000 daltons) are relatively unselective in their target site selection because the relatively small PEG can penetrate into otherwise poorly accessible regions on the protein surface. Alternatively, high molecular weight PEGs may be employed. Such high molecular weight PEGs may have a molecular weight of up to 60,000 daltons. High molecular weight PEGs provide increased linkage chemistry stability and may be beneficial when site-specific pegylation is required. PEG derivatives having a branched structure have a relatively large molecular volume. Accordingly, some advantages of PEG attachment can be obtained without as many points of attachment when using a branched PEG derivative.

Asialo-interferon may be pegylated at a number of different residues within the amino acid sequence, including at a cysteine, lysine, serine, threonine, tyrosine, aspartic acid, or glutamic acid residue; at a C-terminal carboxyl; or at an N-terminal amine of the interferon. For example, asialo-interferon-α-2b having the N-terminal leader removed (amino acids 1-23) may be pegylated at any one of the following positions: Cysteine 1, Lysine 23, Lysine 31, Lysine 49, Lysine 70, Lysine 83, Lysine 112, Lysine 121, Tyrosine 129, Lysine 131, Lysine 133, Lysine 134, and Lysine 164 of the bold sequence shown in FIG. 2A. An asialo-interferon of the invention may be conjugated at one or at multiple sites with a PEG polymer.

The pegylation reaction may be carried out by incubating purified asialo-interferon with an electrophilic derivative of PEG (SC-PEG), or any other activated form of PEG, in 100 mM sodium phosphate at pH 6.5 prior to separating the reaction product by ion exchange chromatography. Such an ion exchange column may be an SP-5PW strong cation exchange column (21.5 mm i.d., 15 cm length, 13 μm particle size, Toso Haas, Montgomeryville, Pa.). The column may be equilibrated in 10 mM sodium phosphate buffer at pH 5.8 and the pegylated product may be eluted using increasing percentages of 80 mM sodium phosphate buffer at pH 5.8 and detected using UV light at a wavelength of 214 nm. To concentrate the isolated product, a CENTRIPLUS- 10 micro-concentrator column (Amicon, Beverly, Mass.) with a molecular mass cutoff of 10 kDa may be used.

Alternatively, an asialo-interferon may be pegylated by incubating a mixture of asialo-interferon with PEG in a 1:3 molar ratio in 50 mM sodium borate buffer at pH 9.0. The final protein concentration of this mixture may be approximately 5 mg/ml. The reaction mixture can then be stirred for 2 hours at 4° C. and the reaction can be stopped by adjusting the pH of the mixture to 4.5 with glacial acetic acid. To isolate the desired reaction product, the mixture can be diluted 10-fold in water and applied onto a column packed with Fractogel® EMD CM 650(M) methacrylate-based polymeric hydrophilic chromatographic resin that has been previously equilibrated with 20 mM sodium acetate (pH 4.5), at a linear velocity of 1.3 cm/min. Protein can be loaded onto a column at a concentration of 2 mg/ml. The column can be washed with the equilibration buffer to remove excess PEG reagent and reaction byproducts. The desired pegylated asialo-interferon may be eluted from the column with 200 mM sodium chloride in the equilibration buffer. The purified pegylated product may be further concentrated and stored in a sterile buffer containing 20 mM sodium acetate (pH 5.0) and 150 mM sodium chloride at 4° C.

Furthermore, positional isomers may be distinguished by using a Waters Delta Prep 3000 preparative HPLC system (Analytical Sales and Service, Mahwah, N.J.) equipped with an SP-5PW strong cation exchange column (e.g., Toso Haas, 21.5 mm i.d., 15 cm length, 13 μm particle size, or 7.5 mm i.d., 75 mm length, 10 μm particle size) at a flow rate appropriate for the column (e.g., 6 mL/min for the 21.5 mm i.d. column and 1 mL/min. for the 7.5 mm i.d. column). These columns may be run with a linear gradient of increasing sodium phosphate concentrations (pH 5.8), or a linear ascending pH gradient (4.3-6.4) from 0 to 100% of potassium phosphate, dibasic (pH 6.4). The positional isomers may be detected using UV light at a wavelength of 214 nm or 280 nm.

Preparation of Other Modified Asialo-Interferons

In addition to the pegylated asialo-interferons described above, other water-soluble polymers may also be conjugated to asialo-interferons. Furthermore, a single interferon may be modified by more than one type of water soluble polymer. For example, an interferon may be conjugated with a PEG and a PVP polymer. Examples of suitable water-soluble polymers include polyvinylpyrrolidone (PVP), poly(vinyl alcohol) (PVA), poly(alkylene oxides) such as poly(propylene glycol) (PPG), polytrimethylene glycol (PTG), and poly(oxyethylated polyols) such as poly(oxyethylated sorbitol), poly(oxyethylated glycerol), and poly(oxyethylated glucose). Such polymers are commercially available, for example, from Sigma-Aldrich (St. Louis, Mo.). Furthermore, water-soluble polymers may be activated prior to conjugation to an asialo-interferon.

Techniques for activating polymers prior to protein conjugation are known in the art. For example, the mPEG derivatives described above are activated forms of PEG. The activation of hydroxyl groups may be accomplished using trichloro-s-triazine (TsT; cyanuric acid). Alternatively, hydroxyl groups may be activated through formation of an amine reactive N-hydroxyl succinimidyl- or p-nitrophenyl carbonate active ester (see, for example, Zalipsky et al., Biotechnol. Appl. Biochem. 15:100-114, 1992). In addition, activation may be achieved when a hydroxyl-containing polymer is first reacted with a cyclic anhydride (e.g., succinic or gluraric anhydride) and followed by coupling the carboxyl modified product of this reaction with N-hydroxyl succinimide in the presence of carbodiimides. This reaction results in succinimidyl succinate or glutarate-type active esters (Abuchowski et al., Cancer Biochem. Biophys. 7:175-186, 1984). Activation may also be achieved through the formation of an imidazolyl carbamate intermediate by reacting the polymer with N,N′-carbonyldiimidazole (CDI). A CDI-activated polymer reacts with amine groups of a protein to form a stable N-alkyl carbamate linkage identical to that formed by using succinimidyl carbonate chemistry (Beauchamp et al., Anal. Biochem. 131:25-33, 1983).

Any of the polymers described herein may be conjugated to an asialo-interferon. In general, polymers may be covalently attached, either with or without prior activation, to proteins via pendant groups that are present in an asialo-interferon or that have been added to the asialo-interferon using chemical modification or other standard methods. Examples of such pendant groups include primary amino groups, carboxyl groups, aromatic rings, and thiol groups. Desirable groups for coupling a polymer to an asialo-interferon include, for instance, the free amino groups in lysine residues present in the protein and the a-amino group of the N-terminal amino acid.

The ratio of polymer to protein to be used in carrying out the conjugation reaction depends on the characteristics (e.g., structure, size, charge, and reactivity) of the polymer as well as the characteristics of the subunit to which the polymer is to be coupled. Determining this ratio is a matter of routine experimentation, for example, by varying the ratio and determining the biological activity (e.g., anti-proliferative or anti-viral activity, as described in the next section) and conjugate stability of the reaction product.

Assaying the Biological Activity of a Modified Asialo-Interferon

Many standard methods in the art may be used to assay the anti-viral and anti-proliferative activity of a modified asialo-interferon, such as a pegylated asialo-interferon (e.g., the methods disclosed in Monkarsh et al., Analytical Biochemistry 247:434-440, 1997 and Bailon et al., Bioconjugate Chem. 12:195-202, 2001). For example, the anti-viral activity of various modified asialo-interferon isomers may be determined in a microtiter plate assay as described in Grace et al. (J. of Interferon and Cytokine Research 21:1103- 1115, 2001). In such an assay, mammalian cells susceptible to viral infection such as Mardin-Darby bovine kidney cells or human foreskin fibroblast cells, are infected with a virus, e.g., vesicular stomatitis virus or encephalomyocarditis virus. The relative potency of modified asialo-interferon can then be determined by comparing the dose of the test modified asialo-interferon which affords 50% protection from a viral cytopathic effect to infected cells with the dose of a control interferon (e.g., interferon-α2a, asialo-interferon, or a reference pegylated asialo-interferon).

In addition, animal models may be used to assay the anti-neoplastic activity of a modified asialo-interferon. For example, athymic nude mice may be implanted with a cancer cell line such as human renal A498 or human renal ACHN cells. In particular, 2×10⁶ cells may be implanted subcutaneously under the rear flank of the mouse. The cells are then given three to six weeks to establish a tumor having an approximate size of 0.05 to 0.50 cubic centimeters. The mice can be treated at least once weekly with a test dosage of modified asialo-interferon. The treatment regimen may last four to five weeks. After treatment, the change in tumor size is compared between the treatment group and a control group, for example, one receiving interferon-α2a or interferon-β1a, and the relative anti-neoplastic activity of the modified asialo-interferon may be assessed in this manner.

Alternatively, the anti-proliferative activity of a modified asialo-interferon may be assayed by using a cell culture assay. For example, human Daudi cells (a Burkitt's lymphoma) maintained in a stationary suspension culture in RMPI 1640 supplemented with 15% fetal bovine serum and 2 mM glutamine (all available from Grand Island Biologicals, Grand Island, N.Y.) may be used in such an assay. 2×10⁴ cells may be added to each well of a microtiter plate (Costar, Mass.) in 100 μl of medium. The plates may then be incubated at 37° C. in 5% CO₂ for 72 hours. Sixteen hours before harvesting, the cells may be pulsed with 0.25 mCi/well of [³H] thymidine (New England Nuclear, Boston, Mass.). The cells may be harvested onto glass filters and counted in a liquid scintillation counter. Results obtained from cells treated with modified asialo-interferon and with a control interferon can then be compared to determine the relative anti-proliferative and, accordingly, anti-neoplastic activity of a particular modified asialo-interferon. Other biological activities that may be compared between the test and control cells, such as 2′-5′ oligoadenylate synthetase activity, serum neopterin levels, β2-microglobulin expression, as well as natural killer (NK) cell and lymphokine activated killer (LAK) cell assays are disclosed in Grace et al. (J. Interferon Cytokine Res. 21:1103-1115, 2001) and Bailon et al. (Bioconjugate Chem. 12:195-202, 2001).

Pharmacokinetic and Biodistribution of a Modified Asialo-Interferon

A modified asialo-interferon may be characterized by its pharmacokinetic and pharmacodynamic properties by methods known in the art. Pharmacokinetic parameters, such as Cₘₐₓ, Tₘₐₓ, t_{1/2}, AUC(0−∞), and clearance rate may be analyzed. In addition, pharmacodynamic determination of a viral cytopathic effect may be correlated with serum modified asialo-interferon concentrations. Examples of such methods are described, for instance, in Pepinsky et al. (J. Pharmacol. Exp. Ther. 297:1059-1066, 2001) and Bailon et al. (Bioconjugate Chem. 12:195-202, 2001). Furthermore, the tissue distribution of a radio-labeled asialo-interferon may be evaluated to confirm targeting to the liver.

Dosage

With respect to the therapeutic methods of the invention, it is not intended that the administration of modified asialo-interferon to a patient be limited to a particular mode of administration, dosage, or frequency of dosing; the present invention contemplates all modes of administration, including intramuscular, intravenous, intraperitoneal, intravesicular, intraarticular, intralesional, subcutaneous, or any other route sufficient to provide a dose adequate to decrease the number of neoplastic cells or to prevent replication or dissemination of a virus. The compound(s) may be administered to the patient in a single dose or in multiple doses. When multiple doses are administered, the doses may be separated from one another by, for example, one day, two days, one week, two weeks, or one month. For example, a pegylated asialo-interferon may be administered once a week for, e.g., 2, 3, 4, 5, 6, 7, 8, 10, 15, 20, or more weeks. It is to be understood that, for any particular subject, specific dosage regimes should be adjusted over time according to the individual need and the professional judgment of the person administering or supervising the administration of the compositions. For example, the dosage of modified asialo-interferon can be increased if the lower dose does not provide sufficient anti-neoplastic or anti-viral activity. Conversely, the dosage of modified asialo-interferon can be decreased if the neoplasm or the viral infection is cleared from the patient.

While the attending physician ultimately will decide the appropriate amount and dosage regimen, a therapeutically effective amount of a modified asialo-interferon, such as a pegylated or a pvpylated asialo-interferon, may be, for example, in the range of about 0.0035 μg to 20 μg/kg body weight/day or 0.010 μg to 140 μg/kg body weight/week. Desirably a therapeutically effective amount is in the range of about 0.025 μg to 10 μg/kg, for example, about 0.025, 0.035, 0.05, 0.075, 0.1, 0.25, 0.5, 1.0, 1.5, 2.0, 2.5, 3.0, 3.5, 4.0, 5.0, 6.0, 7.0, 8.0, or 9.0 μg/kg body weight administered daily, every other day, or twice a week. In addition, a therapeutically effective amount may be in the range of about 0.05, 0.7, 0.15, 0.2, 1.0, 2.0, 3.0, 4.0, 5.0, 6.0, 7.0, 8.0, 10.0, 12.0, 14.0, 16.0, or 18.0 μg/kg body weight administered weekly, every other week, or once a month. Furthermore, a therapeutically effective amount of modified asialo-interferon may be, for example in the range of about 100 μg/m² to 100,000 μg/m² administered every other day, once weekly, or every other week. In a desirable embodiment, the therapeutically effective amount is in the range of about 1000 μg/m² to 20,000 μg/m², for example, about 1000, 1500, 4000, or 14,000 μg/m² of modified asialo-interferon administered daily, every other day, twice weekly, weekly, or every other week.

Formulation of Pharmaceutical Compositions

The administration of a modified asialo-interferon (e.g., a pegylated or a pvpylated asialo-interferon) compound may be by any suitable means that results in a concentration of the modified asialo-interferon that, combined with other components, has anti-viral or anti-neoplastic properties upon reaching the target region. The compound may be contained in any appropriate amount in any suitable carrier substance, and is generally present in an amount of 1-95% by weight of the total weight of the composition. The composition may be provided in a dosage form that is suitable for parenteral (e.g., subcutaneous, intravenous, intramuscular, or intraperitoneal) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound immediately upon administration or at any predetermined time or time period after administration. The latter types of compositions are generally known as controlled release formulations, which include (i) formulations that create a substantially constant concentration of the modified asialo-interferon within the body over an extended period of time; (ii) formulations that after a predetermined lag time create a substantially constant concentration of the modified asialo-interferon within the body over an extended period of time; (iii) formulations that sustain modified asialo-interferon action during a predetermined time period by maintaining a relatively constant, effective modified asialo-interferon level in the body with concomitant minimization of undesirable side effects associated with fluctuations in the plasma level of the active modified asialo-interferon substance (sawtooth kinetic pattern); (iv) formulations that localize modified asialo-interferon action by, e.g., spatial placement of a controlled release composition adjacent to or in the diseased tissue or organ; (v) formulations that achieve convenience of dosing, e.g., administering the composition once per week or once every two weeks; and (vi) formulations that target modified asialo-interferon action by using carriers or chemical derivatives to deliver the modified asialo-interferon to a particular target cell type. Administration of modified asialo-interferon compounds in the form of a controlled release formulation is especially preferred for modified asialo-interferons having a narrow absorption window in the gastro-intestinal tract or a relatively short biological half-life.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the modified asialo-interferon is formulated with appropriate excipients into a pharmaceutical composition that, upon administration, releases the modified asialo-interferon in a controlled manner. Examples include single or multiple unit tablet or capsule compositions, oil solutions, suspensions, emulsions, microcapsules, molecular complexes, microspheres, nanoparticles, patches, and liposomes.

Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the active modified asialo-interferon(s), the composition may include suitable parenterally acceptable carriers and/or excipients. The active asialo-interferon(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

As indicated above, the pharmaceutical compositions according to the invention may be in a form suitable for sterile injection. To prepare such a composition, the suitable active modified asialo-interferon(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, dextrose solution, and isotonic sodium chloride solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

Controlled Release Parenteral Compositions

Controlled release parenteral compositions may be in form of aqueous suspensions, microspheres, microcapsules, magnetic microspheres, oil solutions, oil suspensions, or emulsions. Alternatively, the active modified asialo-interferon(s) may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutamnine), poly(lactic acid), polyglycolic acid, and mixtures thereof. Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters)) or combinations thereof.

Solid Dosage Forms for Oral Use

Formulations for oral use include tablets containing the active ingredient(s) in a mixture with non-toxic pharmaceutically acceptable excipients, and such formulations are known to the skilled artisan (e.g., U.S. Pat. Ser. Nos.: 5,817,307, 5,824,300, 5,830,456, 5,846,526, 5,882,640, 5,910,304, 6,036,949, 6,036,949, 6,372,218, hereby incorporated by reference). These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and anti-adhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active modified asialo-interferon substance in a predetermined pattern (e.g., in order to achieve a controlled release formulation) or it may be adapted not to release the active modified asialo-interferon substance until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active modified asialo-interferon substance). The coating may be applied on the solid dosage form in a similar manner as that described in Encyclopedia of Pharmaceutical Technology, supra.

The two modified asialo-interferons may be mixed together in the tablet, or may be partitioned. In one example, the first modified asialo-interferon is contained on the inside of the tablet, and the second modified asialo-interferon is on the outside, such that a substantial portion of the second modified asialo-interferon is released prior to the release of the first modified asialo-interferon.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus, or spray drying equipment.

Controlled Release Oral Dosage Forms

Controlled release compositions for oral use may, e.g., be constructed to release the active modified asialo-interferon by controlling the dissolution and/or the diffusion of the active modified asialo-interferon substance.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more of the compounds of the claimed combinations may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the modified asialo-interferon(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

Other Embodiments

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A modified asialo-interferon, comprising an asialo-interferon that is conjugated to a water-soluble polymer having an average molecular weight of approximately 1,000 to 60,000 daltons.

2. The modified asialo-interferon of claim 1, wherein said water-soluble polymer has an average molecular weight of approximately 10,000 to 20,000 daltons.

3. The modified asialo-interferon of claim 1, wherein said modified asialo-interferon is a pegylated asialo-interferon

4. The modified asialo-interferon of claim 3, wherein said pegylated asialo-interferon is pegylated at a cysteine, lysine, serine, threonine, tyrosine, aspartic acid, or glutamic acid residue; at a C-terminal carboxyl; or at an N-terminal amine.

5. The modified asialo-interferon of claim 4, wherein said pegylated asialo-interferon is pegylated at a cysteine residue.

6. The modified asialo-interferon of claim 4, wherein said pegylated asialo-interferon is pegylated at a lysine residue.

7. The modified asialo-interferon of claim 1, wherein said modified asialo-interferon is a pvpylated asialo-interferon.

8. The modified asialo-interferon of claim 7, wherein said pvpylated asialo-interferon is pvpylated at a cysteine, lysine, serine, threonine, tyrosine, aspartic acid, or glutamic acid residue; at a C-terminal carboxyl; or at an N-terminal amine.

9. The modified asialo-interferon of claim 8, wherein said pvpylated asialo-interferon is pvpylated at a cysteine residue.

10. The modified asialo-interferon of claim 8, wherein said pvpylated asialo-interferon is pvpylated at a lysine residue.

11. The modified asialo-interferon of claim 1, wherein said modified asialo-interferon comprises an asialo-interferon-α, an asialo-interferon-β, or an asialo-interferon-γ.

12. The modified asialo-interferon of claim 11, wherein said asialo-interferon is a human asialo-interferon.

13. The modified asialo-interferon of claim 1, wherein the polypeptide sequence of said asialo-interferon comprises an additional cysteine residue compared to the sequence of mature interferon polypeptide.

14. The modified asialo-interferon of claim 13, wherein said cysteine replaces a threonine or serine residue of said mature interferon polypeptide.

15. A pharmaceutical composition comprising a modified asialo-interferon of claim 1, and a pharmaceutically acceptable excipient.

16. The pharmaceutical composition of claim 15, wherein said water-soluble polymer having an average molecular weight of approximately 1,000 to 60,000 daltons.

17. The pharmaceutical composition of claim 15, wherein said water-soluble polymer having an average molecular weight of approximately 10,000 to 20,000 daltons.

18. The pharmaceutical composition of claim 15, wherein said modified asialo-interferon is a pegylated asialo-interferon.

19. The pharmaceutical composition of claim 15, wherein said modified asialo-interferon is a pvpylated asialo-interferon.

20. The pharmaceutical composition of claim 15, wherein said modified asialo-interferon comprises an asialo-interferon-α, an asialo-interferon-β, or an asialo-interferon-γ.

21. The pharmaceutical composition of claim 15, wherein said modified asialo-interferon is a modified human asialo-interferon.

22. A method of treating a patient with a hepatic disorder comprising administering to said patient a therapeutically effective amount of a pharmaceutical composition comprising a mammalian asialo-interferon conjugated to a water-soluble polymer having an average molecular weight of approximately 1,000 to 60,000 daltons.

23. The method of claim 22, wherein said modified asialo-interferon is a pegylated asialo-interferon.

24. The method of claim 22, wherein said modified asialo-interferon is a pvpylated asialo-interferon.

25. The method of claim 22, wherein said hepatic disorder is viral hepatitis, hepatic cancer, or fibrosis of the liver.

26. The method of claim 22, wherein said patient is infected with a hepatitis B virus or a hepatitis C virus.

27. The method of claim 22, wherein said hepatic disorder is diffuse-type hepatocellular carcinoma, febrile-type hepatocellular carcinoma, and cholestatic hepatocellular carcinoma, hepatoblastoma, hepatoid adenocarcinoma, and focal nodular hyperplasia.

28. The method of claim 22, wherein said modified asialo-interferon comprises an asialo-interferon-α, an asialo-interferon-β, or an asialo-interferon-γ.

29. The method of claim 28, wherein said asialo-interferon is a human asialo-interferon.
